(19) United States
(12) Patent Application Publication
(10) Pub. No.: US 2025/0000116 A1
(43) Pub. Date: Jan. 2, 2025

(54) LACTASE ENZYME INFUSED DAIRY PRODUCTS AND PROCESS

(71) Applicant: Mar Vista Labs, LLC, Los Angeles, CA (US)

(72) Inventors: Jeremy Baker Voss, Los Angeles, CA (US); Alex Rahnasto Osborne, Los Angeles, CA (US); Eva Marie Sheridan, Los Angeles, CA (US); Matthew Alexander Zaney, Los Angeles, CA (US)

(21) Appl. No.: 18/217,474

(22) Filed: Jun. 30, 2023

(51) Int. Cl. A23G 1/42; A23G 1/00; C12N 9/24

(52) U.S. Cl. CPC A23G 1/423; A23G 1/002; A23G 1/0026; A23G 1/0066; C12N 9/2402; A23G 2200/02; C12Y 302/01108

(57) ABSTRACT

A method for manufacturing lactase enzyme infused chocolate consists of specific ordered steps wherein enzyme is infused toward the end of the tempering phase to preserve lactase's active enzymatic properties and the chocolate's flavor profile while addressing lactose sensitivity.

### CROSS-REFERENCE TO RELATED APPLICATIONS

The present application is a continuation of U.S. patent application Ser. No. 17/343,661, filed Jun. 9, 2021, which claims priority to U.S. Provisional Application No. 63/036,812, filed Jun. 9, 2020, the disclosures of which are hereby incorporated by reference in their entirety for all purposes.

### FIELD

Embodiments of the present disclosure generally relate to lactase enzyme infused dairy products and process to make lactase enzyme infused food, and more specifically to lactase enzyme infused chocolate and process to infuse lactase enzyme in chocolate.

### BACKGROUND

Lactose sensitivity or intolerance is caused by the body producing an insufficient amount of the lactase enzyme to digest the sugar, lactose, naturally occurring in dairy products and affects around 70% of adults globally.

Lactose is milk sugar and occurs naturally in the milk of animals. Many people are sensitive to milk products because they lack the enzyme called lactase. This enzyme, found in the gastrointestinal tract, is critical in the digestion of lactose. If the lactase enzyme is missing or depleted, the gastrointestinal tract cannot adequately break down the milk sugar, leading to a wide variety of symptoms. When this occurs, these individuals are described as being “lactose sensitive” or “lactose intolerant”.

Lactose sensitivity and intolerance did not develop as conditions, but rather lactose tolerance developed as a genetic mutation for a minority of humans due to milk products' dietary benefits, which include protein, micronutrients, calcium and carbohydrates. The production of cheese, which is lower in lactose than raw milk, is thought to have been partly motivated by the need to lower the food's lactose to a tolerable level.

Despite a sensitivity to lactose that affects around 70% of adults, billions of adults still consume dairy products (products containing the lactose sugar) with over $400B dairy products purchased annually.

Symptoms from lactose intolerance can vary greatly from one individual to the next as well as vary within the individual. These symptoms include but are not limited to the following:
Stomach cramps
Intestinal bloating
Flatulence
Diarrhea
Headaches
Nausea

Current solutions exist to enable lactose-sensitive individuals to consume products containing lactose. However, these solutions have been limited in their reach and impact by several factors. It is disappointing that lactase tablets are the best available solution to tolerating meals containing lactose. The tablets make the consumers feel that they have a medical issue, despite the fact that lactose sensitivity affects around 70% of adults, the chalky taste is undesirable, and the consumers rarely have the tablets when they actually need them, e.g., before eating a meal containing lactose, because the tablets are sold in bulk rather than as a tasty, single-serve product purpose-merchandised adjacent to dairy products.

### SUMMARY

In general, the present disclosure includes lactase enzyme infused chocolate and process to infuse lactase enzyme in chocolate. The chocolate can be a single-serve chocolate. In some embodiments, the present disclosure includes techniques for leveraging chocolate as a delivery mechanism for the lactase enzyme to address lactose sensitivity. A process infuses the lactase enzyme in chocolate while maintaining the enzyme's active properties and without noticeably affecting the chocolate's flavor profile.

In some embodiments, a process for infusing lactase enzyme in chocolate can comprise the steps of determining a measure of raw chocolate, first melting of the raw chocolate based on the determined measure, cooling of the melt chocolate, second heating of the cooled chocolate, and adding a pre-determined amount of enzyme to the heated chocolate. After adding the pre-determined amount of enzyme to the heated chocolate, the process can further include pouring the chocolate into a mold. The process can then include packaging the molded chocolate.

The process can be unique because it infuses the enzyme toward the end of the tempering phase.

This summary and the following detailed description are merely exemplary, illustrative, and explanatory, and are not intended to limit, but to provide further explanation of the invention as claimed. Additional features and advantages of the invention will be set forth in the descriptions that follow, and in part will be apparent from the description, or can be learned by practice of the invention. The objectives and other advantages of the invention will be realized and attained by the structure particularly pointed out in the written description, claims and the appended drawings.

### BRIEF DESCRIPTION OF THE DRAWINGS

The present invention may be better understood by referring to the following figures. The components in the figures are not necessarily to scale. Emphasis instead being placed upon illustrating the principles of the disclosure. In the figures, reference numerals designate corresponding parts throughout the different views.

FIG. 1 shows an example process for infusing lactase enzyme in chocolate, according to some embodiments of the present disclosure.

FIG. 2 shows an example process for packaging the lactase enzyme infused chocolate, according to some embodiments of the present disclosure.

FIG. 3 shows an example diagram of the process of FIG. 1 and FIG. 2 in three general phases, according to some embodiments of the present disclosure.

FIG. 4A shows an example front view of a package of the lactase-infused chocolate offering, according to some embodiments of the present disclosure.

FIG. 4B shows an example back view of a package of the lactase-infused chocolate offering, according to some embodiments of the present disclosure.

FIG. 5A shows an example of a display for packages of the lactase-infused chocolate offering, according to some embodiments of the present disclosure.

FIG. 5B shows another example of a display for packages of the lactase-infused chocolate offering, according to some embodiments of the present disclosure.

FIG. 6 shows a table of the lactose content in some common dairy products.

### DETAILED DESCRIPTIONS

The following disclosure describes various embodiments of the present invention and method of use in at least one of its preferred, best mode embodiments, which is further defined in detail in the following description. Those having ordinary skill in the art may be able to make alterations and modifications to what is described herein without departing from its spirit and scope. While this invention is susceptible to different embodiments in different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

As used herein and in the appended claims, the singular forms “a,” “an,” and “the” include plural referents unless the context clearly dictates otherwise.

In general, terms such as “coupled to,” and “configured for coupling to,” and “secure to,” and “configured for securing to” and “in communication with” (for example, a first component is “coupled to” or “is configured for coupling to” or is “configured for securing to” or is “in communication with” a second component) are used herein to indicate a structural, functional, mechanical, electrical, signal, optical, magnetic, electromagnetic, ionic or fluidic relationship between two or more components or elements. As such, the fact that one component is said to be in communication with a second component is not intended to exclude the possibility that additional components may be present between, and/or operatively associated or engaged with, the first and second components.

In the following description and in the figures, like elements are identified with like reference numerals. The use of “e.g.,” “etc.,” and “or” indicates non-exclusive alternatives without limitation, unless otherwise noted. The use of “including” or “includes” means “including, but not limited to,” or “includes, but not limited to,” unless otherwise noted.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

Provided herein are example embodiments of a system and process to address lactose sensitivity. In some embodiments, the system and process of the present disclosure can leverage chocolate as a delivery mechanism for the lactase enzyme to address lactose sensitivity. For example, the embodiments can include a manufacturing process for including the lactase enzyme in chocolate and preserving its active enzymatic properties in addressing lactose sensitivity. The present disclosure can achieve this without denaturing the lactase enzyme by implementing a process that heats, cools, re-heats and infuses the lactase enzyme at specific temperatures and intervals, and in a specific order. As a result, the process can include the lactase enzyme in chocolate to address lactose sensitivity while preserving lactase's active enzymatic properties and the chocolate's flavor profile.

Most chocolate is created through a process that involves taking raw cacao beans, processing them into cacao powder, mixing with other ingredients into a liquid paste, tempering the mixture, pouring the mixture into a mold and prepping the finished chocolate for final distribution. This process is for generically flavored chocolate. However, it will not work with our enzymes because during the initial steps of the tempering process, the increase in temperature to melt the sugars (e.g., 116 degrees Fahrenheit) renders the enzymes ineffective. The result is a chocolate that has included enzymes, but none with any medical benefits.

The system and process of the present disclosure is unique because they infuse the enzyme toward the end of the tempering phase, instead of before. This successfully preserves the enzymatic activity of lactase and maintains the desired flavor profile of the chocolate, for example, without discernibly changing the taste profile of the chocolate.

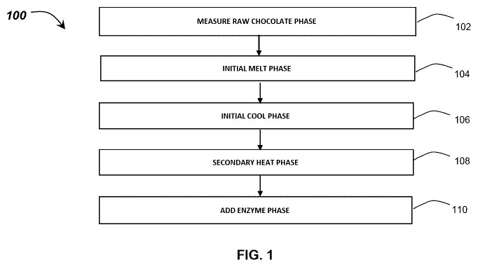

The process of the present disclosure can mix enzymes into chocolate during a secondary heating phase of the tempering process (see FIG. 1, below). After this point, the chocolate is never above 90 degrees, an extremely safe temperature to preserve enzymatic activity. The result is a chocolate that is high quality, with a strong flavor profile, infused with tasteless enzymes that address lactose sensitivity.

The difficulty and non-obvious nature of this process is illustrated by the fact that we experimented with several other delivery mechanisms, including gummies and chewables, but failed due to lower melting points and other properties that prevented us from preserving the active properties of the lactase enzyme through the formulation process. Additionally, we investigated adding lactase at other points in the tempering phase before successfully finding that Step 5 below (“Add Enzyme”), was the right one.

### Process

To make the lactase-infused chocolate of the present disclosure, one would need to follow the formulation process as disclosed herein step-by-step. Changing the ordering of the steps, or the degrees to which the process heats or cools the chocolate, or the phase in which the process adds the enzyme, would likely result in significant problems, including but not limited to irregular texture of the material, uneven distribution of the lactase enzyme, or a degrading of the enzyme at temperatures at which the enzyme naturally denatures.

The embodiments of the present disclosure focus on a key component: the formulation process of the lactase-infused chocolate.

As shown in FIG. 1, in some embodiments, the present disclosure includes an example process 100 for infusing lactase enzyme in chocolate that can comprise the steps of determining a measure of raw chocolate, first melting of the raw chocolate based on the determined measure, cooling of the melt chocolate, second heating of the cooled chocolate, and adding a pre-determined amount of enzyme to the heated chocolate. After adding the pre-determined amount of enzyme to the heated chocolate, the process can further include pouring the chocolate into a mold. The process can then include packaging the molded chocolate.
1. Measure Raw Chocolate
2. Initial Melt
3. Initial Cool
4. Secondary Heat
5. Add Enzyme
6. Pour into Mold
7. Packing Phase

In the Raw Chocolate phase, raw cacao can be transformed into powder and the flavoring can be added to the formula to create a chocolate paste. In the Measure Raw Chocolate phase (Step 102), the chocolate paste can be measured to a desired or predetermined amount, which can then be put into a large pot.

In the Initial Melt phase (Step 104), the chocolate can be heated to melt. For example, the chocolate can be heated to 116-120 degrees Fahrenheit.

In the Initial Cool phase (Step 106), the chocolate can be cooled to the low 80-degrees Fahrenheit (e.g., 80-85 degrees F.). For example, the chocolate can be cooled in a water jacketed pot.

In the Secondary Heat phase (Step 108), once the chocolate is cooled, the chocolate can be re-heated. For example, the chocolate can be re-heated to 88-90 degrees Fahrenheit and mixed.

In the Add Enzyme phase (Step 110), during the secondary heating process, enzyme can be added into the chocolate. Using constant churning, the enzyme can mix evenly into the chocolate. Other suitable mixing materials or instruments used to distribute the enzyme evenly in the chocolate can also be used.

It should be noted that the instruments used to achieve desired temperatures (e.g., the water jacketed pot used to cool the chocolate) can be of other suitable materials.

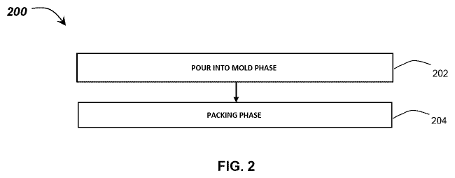

As shown in FIG. 2, in some further embodiments, in the Pour into Mold phase (Step 202), after the chocolate from Step 110 is heated, it can be poured into a desired single-serve mold. For example, the chocolate can be heated to 90 degrees Fahrenheit. The measurement that results in the single-serve chocolate can vary.

In the Packing phase (Step 204), the final formulation is allowed to cool and processed into its production shape, size and packaging.

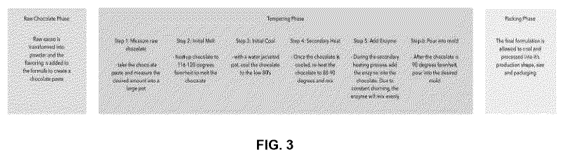

FIG. 3 shows an example diagram of the process of FIG. 1 and FIG. 2 in three general phases: Raw Chocolate phase, Tempering phase and Packaging phase.

### Examples Implementations

In some implementations, using the lactase-infused chocolate to solve lactose sensitivity can be simple: Eat the lactase-infused chocolate prior to consuming a food containing lactose sugar. The lactase-infused chocolate can deliver the lactase enzyme that the lactose-sensitive individual does not produce a sufficient amount of, enabling the person to successfully break down the lactose sugar in the food they consume, and therefore avoid the indigestion, nausea, discomfort, bloating, or other symptoms that may have otherwise resulted. It can be optimal to eat the lactase-infused chocolate as soon as possible before any meal containing the lactose sugar. For example, the effects of the ingested lactase generally diminish or start to diminish within 30 to 45 minutes.

There can also be several possible alternatives or enhancements to the implementations to effectively, conveniently, or desirably deliver chocolate infused with the lactase enzyme.

In some implementations, the delivery mechanism can be something convenient or tailor-made for the desired dairy product other than single-serve chocolate. Examples can include lactase-infused chocolate sprinkles for ice cream, chocolate spice or flavoring or non-dairy creamer for coffee, a pizza topping infused with lactase, and so on.

In some implementations, including additional ingredients in the lactase-infused chocolate can further aid in digestion of the meal being consumed, addressing other components of dairy such as carbohydrates or protein digestion, or targeting other common foods such as starches or pectin. Examples of other value-add ingredients can include probiotics, cellulase, lipase, protease, peptidase, alpha-galactosidase, pectinase, amylase and more.

In some implementations, the product of the present disclosure can include a one-size-fits-all 6-gram chocolate infused with 10K FCC lactase units. However, the lactase-infused chocolate offerings can have varying quantities of the lactase enzyme for individuals who suffer from varying degrees of lactose sensitivity, and/or for meals that contain wide ranges of lactose.

In one example implementation, a single-serve chocolate infused with lactase of the present disclosure was sold at ice cream, pizza, cheese, smoothie, coffee, and grocery stores. The product received a strong response from store owners and customers, with stores selling the product quickly and customers responding positively to an offering that delivered lactase in a tasty, single-serve chocolate where they needed it most-adjacent to dairy products or at the point-of-sale.

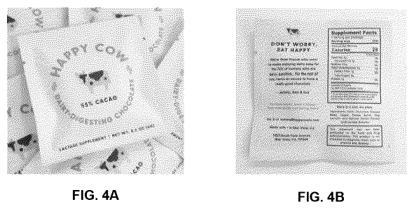

FIG. 4A shows an example front view of a package of the lactase-infused chocolate offering of the present disclosure. FIG. 4B shows an example back view of a package of the lactase-infused chocolate offering of the present disclosure being placed near some dairy products.

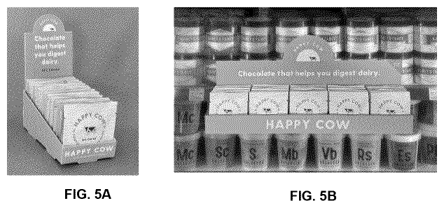

FIG. 5A shows an example of a display for packages of the lactase-infused chocolate offering of the present disclosure. FIG. 5B shows another example of a display for packages of the lactase-infused chocolate offering of the present disclosure.

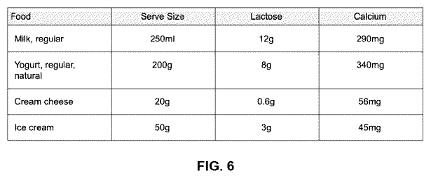

FIG. 6 shows a table of the lactose content in some common dairy products.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments can be recited in or added to the claims, as well as negative limitations that define the inventive scope of the claims by features, functions, steps, or elements that are not within that scope.

It is to be understood that this disclosure is not limited to the particular embodiments described herein, as such may, of course, vary. It is also to be understood that the terminology used herein is for the purpose of describing particular embodiments only and is not intended to be limiting.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

What is claimed is:

1. A method for manufacturing lactase enzyme infused chocolate, the method consisting of the following ordered steps:
transforming raw cacao into powder form to create a chocolate paste;
measuring and depositing a predetermined amount of chocolate paste into a pot;
heating the chocolate;
cooling the heated chocolate;
heating and mixing the cooled chocolate; and
during the heating and mixing step, adding a predetermined amount of enzyme.

2. The method of claim 1, further consisting of the following steps:
pouring the mixed chocolate with enzyme into a desired single-server mold; and
allowing the chocolate to cool in the single-server mold.

3. The method of claim 1, wherein the heating the chocolate step includes heating the chocolate to 116-120 degrees Fahrenheit.

4. The method of claim 1, wherein the cooling the heated chocolate step includes cooling the heated chocolate to the low 80-degrees Fahrenheit.

5. The method of claim 1, wherein the heating and mixing the cooled chocolate step includes heating and mixing the cooled chocolate to 88-90 degrees Fahrenheit.

6. The method of claim 1, wherein the adding a predetermined amount of enzyme includes constant churning of the chocolate.

7. The method of claim 1, wherein the cooling the heated chocolate step includes cooling the chocolate in a water jacketed pot.

8. A lactase enzyme infused chocolate, the lactase enzyme infused chocolate is manufactured by the following ordered steps:
transforming raw cacao into powder form to create a chocolate paste;
measuring and depositing a predetermined amount of chocolate paste into a pot;
heating the chocolate;
cooling the heated chocolate;
heating and mixing the cooled chocolate; and
during the heating and mixing step, adding a predetermined amount of enzyme.

9. The lactase enzyme infused chocolate of claim 8 is further manufactured by the steps of:
pouring the mixed chocolate with enzyme into a desired single-server mold; and
allowing the chocolate to cool in the single-server mold.

10. The lactase enzyme infused chocolate of claim 8, wherein the heating the chocolate step includes heating the chocolate to 116-120 degrees Fahrenheit.

11. The lactase enzyme infused chocolate of claim 8, wherein the cooling the heated chocolate step includes cooling the heated chocolate to the low 80-degrees Fahrenheit.

12. The lactase enzyme infused chocolate of claim 8, wherein the heating and mixing the cooled chocolate step includes heating and mixing the cooled chocolate to 88-90 degrees Fahrenheit.

13. The lactase enzyme infused chocolate of claim 8, wherein the adding a predetermined amount of enzyme includes constant churning of the chocolate.

14. The lactase enzyme infused chocolate of claim 8, wherein the cooling the heated chocolate step includes cooling the chocolate in a water jacketed pot.
